(19) United States
(12) Patent Application Publication
(10) Pub. No.: US 2022/0413273 A1
(43) Pub. Date: Dec. 29, 2022

(54) LASER SYSTEM AND COMPONENTS OF SAME

(71) Applicant: LISA Laser Products GmbH, Katlenburg-Lindau (DE)

(72) Inventors: Samir Lamrini, Goettingen (DE); Thorsten Muehlke, Goettingen (DE); Bjoern Christoph, Bodensee (DE)

(21) Appl. No.: 17/378,569

(22) Filed: Jul. 16, 2021

Related U.S. Application Data
(60) Provisional application No. 63/215,052, filed on Jun. 25, 2021.

(51) Int. Cl. G02B 19/00; H01S 3/0941; H01S 3/16; H01S 3/06; H01S 3/042; H01S 3/02

(52) U.S. Cl. CPC G02B 19/0014; H01S 3/09415; H01S 3/1603; H01S 3/1643; H01S 3/0602; H01S 3/042; H01S 3/025; G02B 19/0052

(57) ABSTRACT

A laser system includes a laser diode that, upon activation, selectively produces a continuous wave of laser light or uniformly spaced, intermittent pulses of laser light. The system further includes a laser focuser with a plurality of lenses that focus the laser light produces by the laser diode and direct the laser light to an optical resonator. The optical resonator includes a lasing medium that, when intersected by the laser light from the laser diode, produces a beam of laser light with a wavelength that may be used for therapeutic treatment. The system is operable to produce the therapeutic laser light when the laser diode is operating in either the continuous wave mode or the pulsed mode, without moving components of the system relative to one another.

### BACKGROUND

### Technical Field

The present disclosure relates to medical devices. More specifically, the disclosure relates to a laser system, components thereof, and methods operating the laser system.

### Description of the Related Art

Lasers are known for use in a number of medical applications. One example is the use of lasers to break up human stones. Human stones may develop within a human body and cause symptoms, such as pain. One type of human stone is a kidney stone. Kidney stone disease, also known as urolithiasis, is when a solid piece of material (kidney stone) develops in the urinary tract. Kidney stones typically form in the kidney and leave the body in the urine stream. A small kidney stone may pass without causing symptoms, however if a kidney stone grows to more than 5 millimeters, it can cause blockage of the ureter, resulting in severe pain. Larger human stones may require procedures such as ureteroscopy for removal.

Ureteroscopy is a procedure in which a urologist positions an endoscope proximate a target area for treatment within a patient's body. Using a laser, the urologist fragments the kidney stone into smaller pieces and retracts the fragments with a basket. Known ureteroscopy treatment utilizes a holmium, e.g., a Holmium:yttrium-aluminium-garnet (Ho:YAG), laser to break up kidney stone fragments in a procedure known as lithotripsy.

### BRIEF SUMMARY

Lasers used in a medical procedure (e.g., lithotripsy) may operate in a continuous wave mode or a pulsed mode. In pulsed mode lasers produce a pulsed beam of laser light that includes intervals of high peaks of power separated by intervals of relatively low (or no) power. For example, a holmium-based laser may operate at 30 Hertz (Hz), such that the beam of light produced over one second includes 30 peaks of power separated by 30 intervals of low (or no) power of equal length.

In continuous wave mode, rather than regularly spaced intervals of peaks of high power spaced by intervals of low (or no) power, the laser maintains a steady output of power over an amount of time (e.g., a second or greater) until the laser producing the continuous wave is affirmatively deactivated (e.g., by a user or a controller). A continuous wave laser may be activated and deactivated, but the intervals between such activations are not necessarily of equal length.

Each of the continuous wave mode and the pulsed mode have respective advantages and disadvantages (e.g., during one or more treatment procedures). For example, a laser operating in pulsed mode may enable dusting of larger human stones than the same laser operating in continuous wave. Additionally, a pulsed mode may result in less heat load being transferred to surrounding tissues and less carbonization of those tissues. However, the pulse energy of some lasers (e.g., holmium-based lasers) may be high enough to cause retropulsion (i.e., movement) of human stones that are impacted by a beam of light created by a holmium-based laser. For example, a Holmium:yttrium-aluminium-garnet (Ho:YAG) laser produces a beam of light with a wavelength of 2,100 nanometers (nm). A laser operating in continuous wave mode, on the other hand, may result in less energy being needed for procedures involving soft tissue to achieve the same therapeutic result as would be required to operate the laser in a pulsed mode.

Accordingly, it may be beneficial to provide a single laser system that is operable in both a continuous wave mode and a pulsed mode.

According to one aspect of the disclosure, a laser focuser includes a first lens, a second lens, a third lens, and a fourth lens each having a respective optical axis. The second lens is positioned with respect to the first lens such that the first optical axis and the second optical axis are collinear. The third lens is positioned with respect to the first lens and the second lens such that the first optical axis and the third optical axis are collinear, and the second lens is between the first lens and the third lens. The fourth lens is positioned with respect to the first lens and the third lens such that the first optical axis and the fourth optical axis are collinear, and the third lens is between the first lens and the fourth lens. A first distance measured from the first lens to the second lens along the first optical axis is greater than a second distance measured from the second lens to the fourth lens along the first optical axis.

According to one aspect of the disclosure an optical resonator includes a first mirror, a laser crystal, a lens, and a second mirror. The first mirror has a first surface that is transmissive of light with a first wavelength, and the first mirror has a second surface that is reflective of light with a second wavelength. The laser crystal is positioned with respect to the first mirror such that a longitudinal axis of the laser crystal intersects the first mirror. The lens is positioned with respect to the first mirror and the laser crystal such that the longitudinal axis of the laser crystal intersects the lens and the laser crystal is between the first mirror and the lens. The second mirror has a third surface that reflects a majority of light with the second wavelength that contacts the first surface and transmits a portion of the light with the second wavelength that contacts the first surface through the third surface. A first distance measured from the laser crystal to the lens along the longitudinal axis is greater than a second distance measured from the lens to the second mirror along the longitudinal axis.

According to one aspect of the disclosure, a laser crystal cooling chamber includes an inner wall and an outer wall. The inner wall forms an inner cavity that receives a laser crystal. The inner cavity has a first opening formed in a first terminal end of the cooling chamber and a second opening formed in a second terminal end of the cooling chamber, wherein the cooling chamber has a cooling chamber length that is measured from the first terminal end to the second terminal end along a longitudinal axis of the cooling chamber that passes through both the first opening and the second opening. The outer wall at least partially encloses the inner wall such that an outer cavity is formed between the inner wall and the outer wall. The outer wall forms at least two entry openings that provide passage through the outer wall into the outer cavity, and the outer wall forms at least two exit openings that provide passage through the outer wall out of the outer cavity. Each of the at least two entry openings are positioned closer to the first terminal end than the at least two entry openings are from the second terminal end, and each of the at least two exit openings are positioned closer to the second terminal end than the at least two exit openings are from the first terminal end.

According to one aspect of the disclosure, a laser system includes a laser diode, the laser focuser as described above, the optical resonator as described above, and the laser crystal cooling chamber as described above, wherein the laser focuser is positioned between the laser diode and the optical resonator, and the laser crystal is positioned within the inner cavity.

### BRIEF DESCRIPTION OF THE SEVERAL VIEWS OF THE DRAWINGS

In the drawings, identical reference numbers identify similar elements or acts. The sizes and relative positions of elements in the drawings are not necessarily drawn to scale. For example, the shapes of various elements and angles are not necessarily drawn to scale, and some of these elements may be arbitrarily enlarged and positioned to improve drawing legibility. Further, the particular shapes of the elements as drawn, are not necessarily intended to convey any information regarding the actual shape of the particular elements, and may have been solely selected for ease of recognition in the drawings.

FIG. 1 is a side, elevation, schematic view of a therapeutic laser system, according to an embodiment.

FIG. 2 is a side, elevation view of a source laser of the therapeutic laser system illustrated in FIG. 1, according to an embodiment.

FIG. 3 is a side, elevation, schematic view of a laser focuser of the therapeutic laser system illustrated in FIG. 1, according to an embodiment.

FIG. 4 is a side, cross-sectional view of a first lens of the laser focuser illustrated in FIG. 3.

FIG. 5 is a top, cross-sectional view of the first lens of the laser focuser illustrated in FIG. 3.

FIG. 6 is a side, cross-sectional view of a second lens of the laser focuser illustrated in FIG. 3.

FIG. 7 is a top, cross-sectional view of the second lens of the laser focuser illustrated in FIG. 3.

FIG. 8 is a side, cross-sectional view of a third lens of the laser focuser illustrated in FIG. 3.

FIG. 9 is a top, cross-sectional view of the third lens of the laser focuser illustrated in FIG. 3.

FIG. 10 is a side, cross-sectional view of a fourth lens of the laser focuser illustrated in FIG. 3.

FIG. 11 is a top, cross-sectional view of the fourth lens of the laser focuser illustrated in FIG. 3.

FIG. 12 is a side, cross-sectional view of an optical resonator and a cooling chamber of the therapeutic laser system illustrated in FIG. 1, according to an embodiment.

FIG. 13 is a side, cross-sectional view of the cooling chamber illustrated in FIG. 12.

FIG. 14 is a front, cross-sectional view of the cooling chamber illustrated in FIG. 13 along line A-A.

### DETAILED DESCRIPTION

In the following description, certain specific details are set forth in order to provide a thorough understanding of various disclosed embodiments. However, one skilled in the relevant art will recognize that embodiments may be practiced without one or more of these specific details, or with other methods, components, materials, etc. In other instances, well-known structures associated with therapeutic laser systems have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is as “including, but not limited to.”

Reference throughout this specification to “one embodiment,” “an embodiment,” or “an aspect of the disclosure” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in this specification and the appended claims, the singular forms “a,” “an,” and “the” include plural referents unless the content clearly dictates otherwise. It should also be noted that the term “or” is generally employed in its broadest sense, that is as meaning “and/or” unless the content clearly dictates otherwise.

Recitation of ranges of values herein are merely intended to serve as a shorthand method of referring individually to each separate value falling within the range including the stated ends of the range, unless otherwise indicated herein, and each separate value is incorporated into the specification as if it were individually recited herein.

Aspects of the disclosure will now be described in detail with reference to the drawings, wherein like reference numbers refer to like elements throughout, unless specified otherwise. Certain terminology is used in the following description for convenience only and is not limiting. The term “plurality”, as used herein, means more than one. The terms “a portion” and “at least a portion” of a structure include the entirety of the structure.

The headings and Abstract of the Disclosure provided herein are for convenience only and do not interpret the scope or meaning of the embodiments.

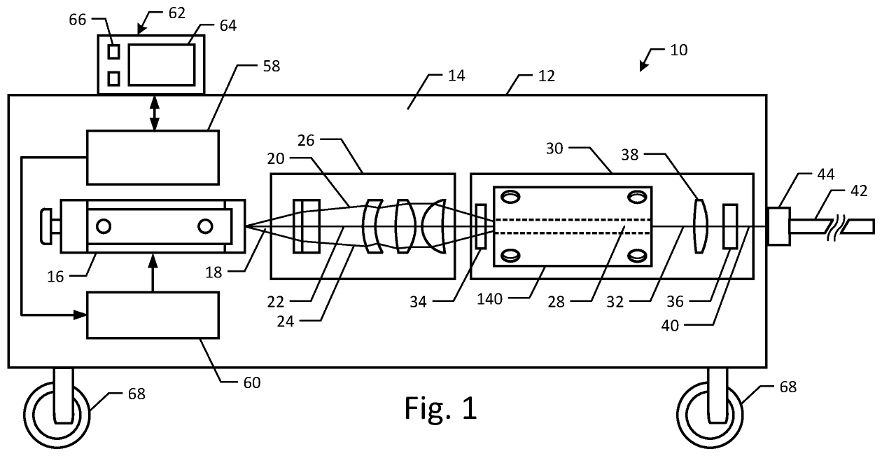

Referring to FIG. 1, a therapeutic laser system 10 may include a housing 12 that selectively encloses an internal cavity 14 formed by the housing 12. The system 10 may include a source laser 16 (e.g., a laser diode). The source laser 16, upon activation, produces a beam of light 18 (or multiple beams of light 18), which includes a plurality of rays of light. In the illustrated embodiment, an upper ray 20, a central ray 22, and a lower ray 24 of the beam of light 18 are shown. The beam of light 18 may have a first wavelength (e.g., between 790 nm to 800 nanometers).

The laser system 10 may include a laser focuser 26 that focuses and directs the beam of light 18 to a lasing medium 28 (e.g., a laser crystal). The lasing medium 28 may be part of an optical resonator 30. The beam of light 18 causes electrons within atoms of the lasing medium 28 to become “excited” and increase their energy level. Once these “excited” electrons return to their “non-excited” ground state (or energy level), energy is released in the form of photons. These photons circulate (i.e., are reflected back and forth through the lasing medium 28) within the optical resonator 30 to form a beam of light 32 with a second wavelength that is different (e.g., longer) than the first wavelength. As shown, the optical resonator may include a first mirror 34 and a second mirror 36 that reflect the beam of light 32 back and forth through the lasing medium 28. The optical resonator 30 may include a lens 38 positioned between the first mirror 34 and the second mirror 36 to redirect and/or focus the beam of light 32 as it travels between the first mirror 34 and the second mirror 36. Typical resonators (e.g., those that use a holmium or thulium doped laser crystal) may be devoid of an intra-cavity lens, such as the lens 38, due to such a lens being unnecessary given the high pulse energy that is used to generate these lasers. The lens 38 may enable stable operation of the optical resonator 30 in both pulsed and continuous wave operating modes.

The first mirror 34 may be a total reflector, which reflects all of the photons of the beam of light 32 with the second wavelength that impact the first mirror 34 back toward the lasing medium 28. The first mirror 34 (e.g., a first surface of the first mirror 34) may be transmissive with respect to the beam of light 18 with the first wavelength, and the first mirror 34 (e.g., a second surface of the first mirror 34) may be reflective with respect to the beam of light 32 with the second wavelength. The second mirror 36 may be a partial reflector, which reflects a portion of the photons of the beam of light 32 that impact the second mirror 36 back toward the lasing medium 28, while allowing a collimated beam of the photons to pass through the second mirror 36, thereby forming a beam of therapeutic laser light 40, which may be directed to exit the therapeutic laser system 10 (e.g., via a waveguide 42 coupled to the housing 12 by a waveguide coupler 44).

According to one embodiment, the lasing medium 28 may be a rare-earth element doped crystal (e.g., a holmium-doped laser crystal). Holmium-based lasers (i.e., beams of light produced by a holmium-doped laser crystal) are known for their use in a number of therapeutic applications. One example of a holmium-based laser is a Holmium:yttrium-aluminium-garnet (Ho:YAG) laser, which produces a beam of light with a wavelength of 2,100 nanometers (nm).

Another rare-earth element doped crystal is thulium:yttrium-aluminium-garnet (Tm:YAG), which produces a focused beam of light with a wavelength of 2,010 nm. A thulium laser may be pumped by laser diodes, which may operate at a higher wall-plug efficiency compared to the flash lamps of a holmium laser, thus resulting in a higher efficiency for a thulium laser. Thulium lasers, however, present challenges related to their engineering and construction. Specifically, the optical focusing design related to a thulium laser used in the therapeutic laser system 10 may be more complex and/or expensive than a holmium laser, as the thulium laser may be operable in both a continuous wave mode and a pulsed mode. Thus, the laser focuser 26 and or the optical resonator 30 of the system 10 may be constructed to each meet the operating criteria for multiple modes of laser operation, as described in detail below. Thulium lasers may produce laser light with a wavelength between 1800 nm and 2200 nm. According to one embodiment, the rare-earth element doped crystal may be co-doped with more than one rare-earth element (e.g., doped with both holmium and thulium).

The system 10 may be selectively operable in both a pulsed mode and a continuous wave mode. In pulsed mode the produced beam of therapeutic laser light 40 is a pulsed beam of laser light that includes intervals of high peaks of power separated by intervals of relatively low (or no) power. For example, a holmium-based laser may operate at 30 Hertz (Hz), such that produced beam of therapeutic laser light 40 over one second includes 30 peaks of power (e.g., each of equal length) separated by 30 intervals of low (or no) power (e.g., each of equal length).

In continuous wave mode the produced beam of therapeutic laser light 40 maintains a steady output of power over an amount of time (e.g., a second or greater) until the system 10 producing the continuous wave is affirmatively deactivated (e.g., by a user or a controller), rather than regularly spaced intervals of peaks of high power spaced by intervals of low (or no) power. A continuous wave laser may be activated and deactivated, but the intervals between such activations are not necessarily of equal length.

The use of a continuous wave of the produced beam of therapeutic laser light 40 may result in less energy being needed to achieve the same therapeutic result as would be required to operate a pulsed beam.

The produced beam of therapeutic laser light 40 produced by the system 10 may be guided to a target. As shown in the illustrated embodiment, the system 10 may include the waveguide 42 (e.g., a laser fiber), with an internal cavity which guides the produced beam of therapeutic laser light 40 along a length of the waveguide 42. The waveguide 42 may include a distal end at which the produced beam of therapeutic laser light 40 exits the internal cavity of the waveguide 42. The waveguide 42 may be flexible so that the distal end is moveable (e.g., relative to a proximal end of the waveguide 42 that is attached to the housing 12) to be positioned adjacent the target.

According to one embodiment, the target may be located within a human body. For example, the target may include one or more urinary stones within a patient's urinary tract. Thus, the system 10 may include an endoscope (e.g., a cystoscope, a ureteroscope, a renoscope, a nephroscope, etc.) and the waveguide 42 may be sized to fit within the endoscope during insertion of the endoscope into the patient's body and advancement to the target.

During advancement of the endoscope and the enclosed waveguide 42, the distal end may be enclosed within an internal cavity of the endoscope, thus protecting the distal end from damage (e.g., due to contact with body tissue). Upon arrival at the target, the waveguide 42 may be advanced within the endoscope such that the distal end is exposed. The advancement of the waveguide 42 may help prevent the produced beam of therapeutic laser light 40 from impacting and potentially damaging the endoscope.

With the distal end of the waveguide 42 pointed at the target, activation of the laser system 10 results in the produced beam of therapeutic laser light 40 impacting the target. According to one embodiment, the target may include a human stone (e.g., a urinary stone), and sustained impact of the produced beam of therapeutic laser light 40 with the human stone results in the human stone breaking into multiple fragments, which due to their smaller size are easier to remove from the patient's body.

The system 10 may further include a controller 58 communicatively coupled to the source laser 16 (e.g., via a power source 60 of the source laser 16). The controller 58 may receive data related to operation of the laser system 10 (e.g., power output, temperature, characteristics of the target, etc.) and/or input from a user (e.g., via a user interface 62 that includes a display 64, input controls 66, or both) and based on the data and/or input enable or prevent activation of the source laser 16.

The display 64 may show operational parameters of the system 10 including, but not limited to, the status (e.g., activated/not activated, continuous wave mode/pulsed mode, etc.) of the source laser 16, the status of the beam of therapeutic laser light 40, the identification/classification of the target, etc. The input controls 66 may allow a user of the system 10 to change one or more of the operational parameters of the system 10 including, but not limited to, the status (e.g., activated/not activated, continuous wave mode/pulsed mode, etc.) of the source laser 16.

The system 10 may be mobile. As shown, the housing 12 may be mounted on wheels 68 so as to allow a user of the system 10 to change the location of the system 10.

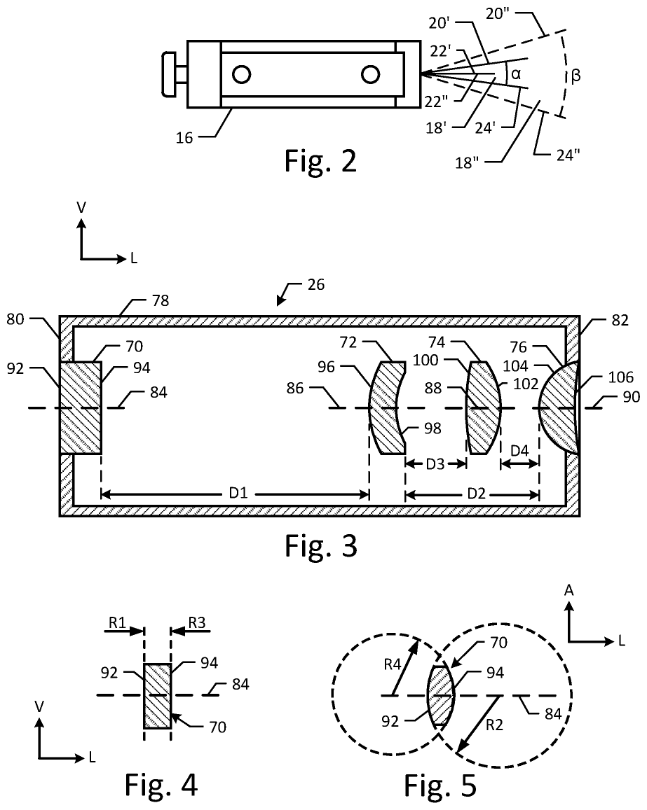

Referring to FIGS. 1 and 2, the system 10 may be operable in multiple modes (e.g., pulsed mode and continuous wave mode). According to one embodiment, the beam of light 18 produced by source laser 16 may have different characteristics dependent on the mode of operation. One characteristic of the beam of light 18 produced by source laser 16 that may change based on the mode of operation is beam divergence. The beam divergence is an angular measurement of the increase in beam diameter with distance from the optical aperture.

As shown in the illustrated embodiment, the source laser 16 when operating in one mode (e.g., a continuous wave mode), produces the beam of light 18′ (depicted by the solid lines). The beam divergence α (alpha) of the beam of light 18′ is measured from the upper ray 20′ (the upper-most ray of the beam of light 18′), through the central ray 22′, and to the lower ray 24′ (the lower-most ray of the beam of light 18′). When the source laser 16 is operating in another mode (e.g., a pulsed mode), the beam of light 18″ (depicted by the dashed lines) is produced. The beam divergence β (beta) of the beam of light 18″ is measured from the upper ray 20″ (the upper-most ray of the beam of light 18″), through the central ray 22″, and to the lower ray 24″ (the lower-most ray of the beam of light 18″). As shown the beam divergence α (alpha) may be different (e.g., less than as shown, or greater than) the beam divergence β (beta).

Referring to FIGS. 1 and 3, the laser focuser 26 focuses and directs the beam of light 18 to the lasing medium 28 regardless of whether the source laser 16 is operating in a pulsed mode or a continuous wave mode. According to one embodiment, the laser focuser 26 may include a plurality of optically powered components (e.g., lenses) arranged in series. As shown, the laser focuser may include a first lens 70, a second lens 72, a third lens 74, and a fourth lens 76. The laser focuser 26 may include a housing 78 that encloses one or more of the first lens 70, the second lens 72, the third lens 74, and the fourth lens 76.

The laser focuser 26 may include a first end 80 through which the beam of light 18 enters the laser focuser 26, and the laser focuser 26 may include a second end 82 through which the beam of light exits the laser focuser 26. The first lens 70 may include a first optical axis 84, the second lens 72 may include a second optical axis 86, the third lens 74 may include a third optical axis 88, and the fourth lens may include a fourth optical axis 90. According to one embodiment, the first lens 70 may be positioned relative to the second lens 72, the third lens 74, and the fourth lens 76 such that the first optical axis 84 is collinear with the second optical axis 86, the third optical axis 88, the fourth optical axis 90, or any combination thereof. As shown the first lens 70, the second lens 72, the third lens 74, and the fourth lens 76 may be arranged such that each of the first optical axis 84, the second optical axis 86, the third optical axis 88, and the fourth optical axis 90 are collinear.

The first lens 70 may be positioned closest to the first end 80 of the first lens 70, the second lens 72, the third lens 74, and the fourth lens 76. The fourth lens 76 may be positioned closest to the second end 82 of the first lens 70, the second lens 72, the third lens 74, and the fourth lens 76. As shown, the second lens 72 may be positioned between the first lens 70 and the third lens 74, and the third lens 74 may be positioned between the second lens 72 and the fourth lens 76.

The first lens 70 may be positioned a first distance D1 from the second lens 72 as measured along a longitudinal direction L, which may be parallel to the first optical axis 84. The second lens 72 may be positioned a second distance D2 from the fourth lens 76 as measured along the longitudinal direction L, and the first distance D1 may be greater than the second distance D2. According to one embodiment, the first distance D1 may be greater than twice the second distance D2. The first distance D1 may be between 43.29 and 47.27 mm, and the second distance D2 may be between 13.57 and 17.54 mm.

The second lens 72 may be positioned a third distance D3 from the third lens 74 as measured along longitudinal direction L. The third lens 74 may be positioned a fourth distance D4 from the fourth lens 76 as measured along longitudinal direction L. According to one embodiment, the third distance D3 may be 1 between and 1.34 mm. According to one embodiment, the fourth distance D4 may be between 0.09 and 0.2 mm.

Referring to FIGS. 3 to 11, according to one embodiment, the first lens 70 may include a first optically powered surface 92 and a second optically powered surface 94 that are opposite one another along the first optical axis 84. As shown, the first optically powered surface 92 may be the surface of the first lens 70 that is closest to and faces towards the first end 80 of the laser focuser 26, and the second optically powered surface 94 may be the surface of the first lens 70 that is closest to and faces towards the second end 82 of the laser focuser 26. According to one embodiment, the first lens 70 may be a 90°-crossed toroidal convex-convex lens.

According to one embodiment, the second lens 72 may include a first optically powered surface 96 and a second optically powered surface 98 that are opposite one another along the second optical axis 86. As shown, the first optically powered surface 96 may be the surface of the second lens 72 that is closest to and faces towards the first end 80 of the laser focuser 26, and the second optically powered surface 98 may be the surface of the second lens 72 that is closest to and faces towards the second end 82 of the laser focuser 26. According to one embodiment, the second lens 72 may be a toroidal convex-concave lens.

According to one embodiment, the third lens 74 may include a first optically powered surface 100 and a second optically powered surface 102 that are opposite one another along the third optical axis 88. As shown, the first optically powered surface 100 may be the surface of the third lens 74 that is closest to and faces towards the first end 80 of the laser focuser 26, and the second optically powered surface 102 may be the surface of the third lens 74 that is closest to and faces towards the second end 82 of the laser focuser 26. According to one embodiment, the third lens 74 may be a toroidal convex-convex lens.

According to one embodiment, the fourth lens 76 may include a first optically powered surface 104 and a second optically powered surface 106 that are opposite one another along the fourth optical axis 90. As shown, the first optically powered surface 104 may be the surface of the fourth lens 76 that is closest to and faces towards the first end 80 of the laser focuser 26, and the second optically powered surface 106 may be the surface of the fourth lens 76 that is closest to and faces towards the second end 82 of the laser focuser 26. According to one embodiment, the fourth lens 76 may be a toroidal convex-concave lens.

As shown in FIGS. 4 and 5, the first optically powered surface 92 may be curved (e.g., convex), and the first optically powered surface 92 may include a first radius of curvature R1 (e.g., a vertical radius of curvature measured within a first plane), and a second radius of curvature R2 (e.g., a horizontal radius of curvature measured within a second plane that is perpendicular to the first plane). According to one embodiment, the first radius of curvature R1 may be infinite (i.e., the first optically powered surface 92 may be cylindrical such that the first optically powered surface 92 is a straight line along the vertical direction V). The use of lenses with cylindrical optically powered surfaces may result in a reduction in complexity of the set up and calibration for the laser focuser 26 as the infinite radius of curvature reduces the precision needed to properly position the lens with respect to at least one degree of freedom. According to one embodiment the second radius of curvature R2 may be between 53.95 and 54.05 mm.

The second optically powered surface 94 may be curved (e.g., convex), and the second optically powered surface 94 may include a third radius of curvature R3 (e.g., a vertical radius of curvature measured within the first plane), and a fourth radius of curvature R4 (e.g., a horizontal radius of curvature measured within the second plane). According to one embodiment the third radius of curvature R3 may be infinite. According to one embodiment the fourth radius of curvature R4 may be between 46.06 and 46.16 mm.

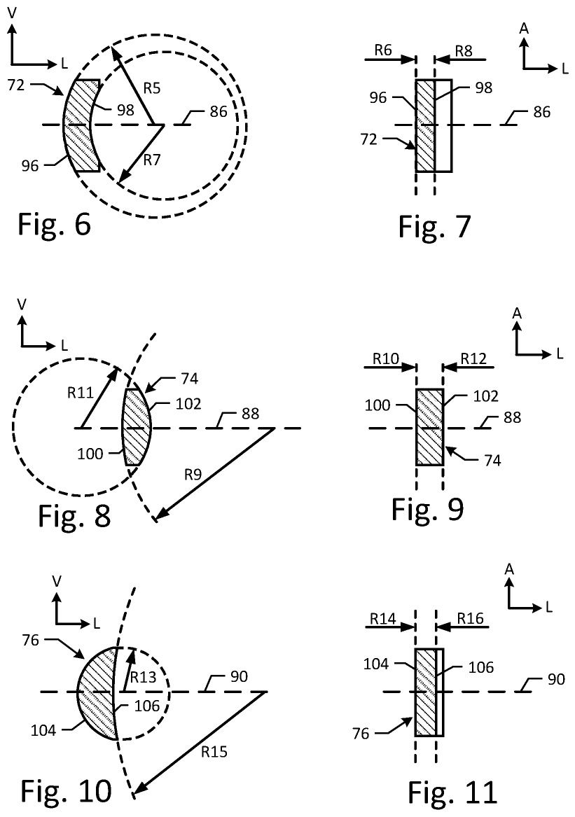

As shown in FIGS. 6 and 7, the first optically powered surface 96 of the second lens 72 may be curved (e.g., convex), and the first optically powered surface 96 may include a fifth radius of curvature R5 (e.g., a vertical radius of curvature measured within the first plane), and a sixth radius of curvature R6 (e.g., a horizontal radius of curvature measured within the second plane. According to one embodiment the fifth radius of curvature R5 may be between 13.64 and 13.74 mm. According to one embodiment the sixth radius of curvature R6 may be infinite. According to one embodiment, the first optically powered surface 96 of the second lens 72 may face towards the second optically powered surface 94 of the first lens 70.

The second optically powered surface 98 of the second lens 72 may be curved (e.g., concave), and the second optically powered surface 98 may include a seventh radius of curvature R7 (e.g., a vertical radius of curvature measured within the first plane), and an eighth radius of curvature R8 (e.g., a horizontal radius of curvature measured within the second plane). According to one embodiment the seventh radius of curvature R7 may be between 9.44 and 9.54 mm. According to one embodiment the eighth radius of curvature R8 may be infinite (i.e., the second optically powered surface 98 may be cylindrical such that the second optically powered surface 98 is a straight line along the vertical direction V). According to one embodiment, the second optically powered surface 98 of the second lens 72 may face towards the first optically powered surface 100 of the third lens 74.

As shown in FIGS. 8 and 9, the first optically powered surface 100 of the third lens 74 may be curved (e.g., convex), and the first optically powered surface 100 may include a ninth radius of curvature R9 (e.g., a vertical radius of curvature measured within the first plane), and a tenth radius of curvature R10 (e.g., a horizontal radius of curvature measured within the second plane. According to one embodiment the ninth radius of curvature R9 may be between 74.21 and 74.31 mm. According to one embodiment the tenth radius of curvature R10 may be infinite. According to one embodiment, the first optically powered surface 100 of the third lens 74 may face towards the second optically powered surface 98 of the second lens 72.

The second optically powered surface 102 of the third lens 74 may be curved (e.g., convex), and the second optically powered surface 102 may include an eleventh radius of curvature R11 (e.g., a vertical radius of curvature measured within the first plane), and a twelfth radius of curvature R12 (e.g., a horizontal radius of curvature measured within the second plane). According to one embodiment the eleventh radius of curvature R11 may be between 17.54 and 17.64 mm. According to one embodiment the twelfth radius of curvature R12 may be infinite. According to one embodiment, the second optically powered surface 102 of the third lens 74 may face towards the first optically powered surface 104 of the fourth lens 76.

As shown in FIGS. 10 and 11, the first optically powered surface 104 of the fourth lens 76 may be curved (e.g., convex), and the first optically powered surface 104 may include a thirteenth radius of curvature R13 (e.g., a vertical radius of curvature measured within the first plane), and a fourteenth radius of curvature R14 (e.g., a horizontal radius of curvature measured within the second plane. According to one embodiment the thirteenth radius of curvature R13 may be between 9.9 and 10 mm. According to one embodiment the fourteenth radius of curvature R14 may be infinite. According to one embodiment, the first optically powered surface 104 of the fourth lens 76 may face towards the second optically powered surface 102 of the third lens 74.

The second optically powered surface 106 of the fourth lens 76 may be curved (e.g., concave), and the second optically powered surface 106 may include a fifteenth radius of curvature R15 (e.g., a vertical radius of curvature measured within the first plane), and a sixteenth radius of curvature R16 (e.g., a horizontal radius of curvature measured within the second plane). According to one embodiment the fifteenth radius of curvature R15 may be between 48.75 and 48.85 mm. According to one embodiment the sixteenth radius of curvature R16 may be infinite (i.e., the second optically powered surface 106 may be cylindrical such that the second optically powered surface 106 is a straight line along the vertical direction V).

According to one embodiment, each of the first lens 70, the second lens 72, the third lens 74, and the fourth lens 76 may be spherical (i.e., each radius of curvature described above remains constant along the respective surface in the respective direction). However, the two radii of any one of the optically powered surfaces of the first lens 70, the second lens 72, the third lens 74, and the fourth lens 76 may be different from one another. For example, the third radius of curvature R3 of the second optically powered surface 94 may be different from (e.g., greater than or less than) the radius of curvature R4 of the second optically powered surface 94.

According to one embodiment, one or more of the first lens 70, the second lens 72, the third lens 74, and the fourth lens 76 may be aspherical (i.e., a radius of curvature described above changes along the respective surface in the respective direction). However, aspherical lenses are typically more expensive to produce, and it may thus result in a more economical laser system 10 if the laser focuser 26 is devoid of aspherical lenses.

One or more of the first lens 70, the second lens 72, the third lens 74, and the fourth lens 76 may be supported within the laser focuser 26 such that one or more of the first distance D1, the second distance D2, the third distance D3, and the fourth distance D4 may be adjusted. According to one embodiment, the first lens 70, the second lens 72, the third lens 74, and the fourth lens 76 may be supported within the laser focuser 26 such that each of the first distance D1, the second distance D2, the third distance D3, and the fourth distance D4 is fixed.

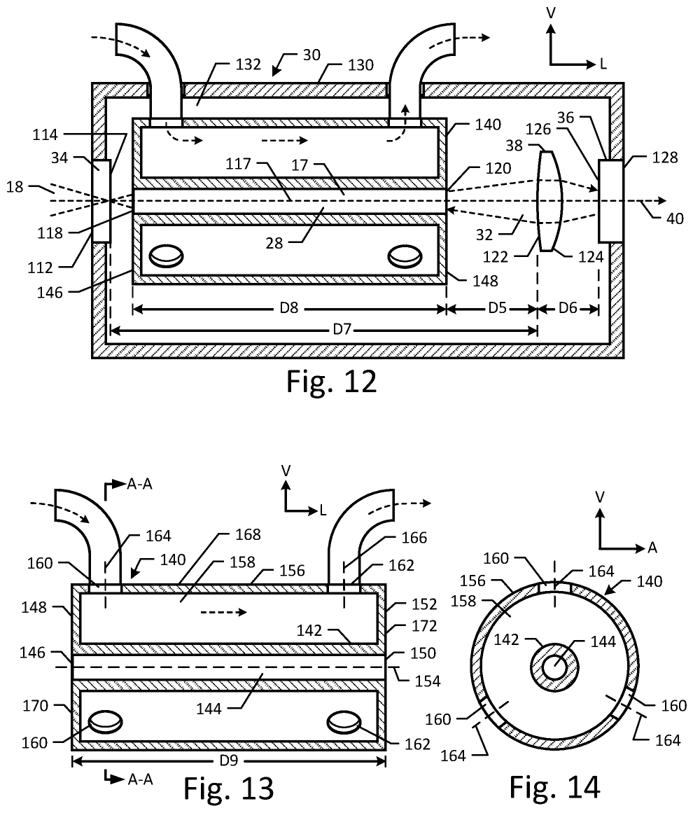

Referring to FIGS. 1 and 12, the optical resonator 30 of the system 10 may include the first mirror 34 having a first surface 112 and a second surface 114. The first surface 112 and the second surface 114 may be opposite one another (e.g., along an optical axis of the first mirror 34), as shown in the illustrated embodiment. The first mirror 34 (e.g., the first surface 112) may be transmissive of the beam of light 18, and the first mirror 34 (e.g., the second surface 114) may be reflective of the beam of light 32. According to one embodiment, the first surface 112 may be transmissive of light with a wavelength within a first range of wavelengths, and may be not transmissive (e.g., reflective) of light with a wavelength that is outside of the first range of wavelengths. The first range of wavelengths may be between 700 nm and 900 nm (e.g., between 790 and 800 nm), according to one embodiment.

According to one embodiment, the second surface 114 may be reflective of light with a wavelength within a second range of wavelengths, and may be not reflective (e.g., transmissive) of light with a wavelength that is outside of the second range of wavelengths. The second range of wavelengths may be between 1800 nm and 2200 nm (e.g., between 1900 and 2100 nm), according to one embodiment.

The lasing medium 28 (e.g., a rare-earth element doped crystal 17) may be elongated along a longitudinal axis 117 that extends through both a first end 118 of the lasing medium 28 and a second end 120 of the lasing medium 28. The lasing medium 28 may be positioned relative to the first mirror 34 such that the beam of light 18, after exiting the first mirror 34, enters the first end 118 of the lasing medium 28. According to one embodiment, the lasing medium 28 may be positioned relative to the first mirror 34 such that the longitudinal axis 117 intersects the first mirror 34 (e.g., is collinear with an optical axis or central axis of the first mirror 34).

The lens 38 of the optical resonator 30 may be positioned relative to the lasing medium 28 such that the beam of light 32, after exiting the second end 120 of the lasing medium 28, enters the lens 38. According to one embodiment, the lens 38 may be positioned relative to the lasing medium 28 such that the longitudinal axis 117 intersects the lens 38 (e.g., is collinear with an optical axis or central axis of the lens 38). The lens 38 may include a first optically powered surface 122 that faces towards the lasing medium 28 and a second optically powered surface 124 that faces towards the second mirror 36. According to one embodiment, the first optically powered surface 122 is a convex surface with a vertical radius of curvature of about 355 mm. According to one embodiment, the second optically powered surface 124 is a convex surface with a vertical radius of curvature of about 58 mm.

The second mirror 36 may be positioned relative to the lens 38 such that the beam of light 32, after exiting the second optically powered surface 124, of the lens 38, intersects the second mirror 36. According to one embodiment, the second mirror 36 may be positioned relative to the lens 38 such that a central axis of the second mirror 36 is collinear with an optical axis or central axis of the lens 38. According to one embodiment, the second mirror 36 may include a first surface 126 that reflects a majority of the beam of light 32 (e.g., light with the second wavelength) back toward the lens 38 to be redirected to the first mirror 34 and then back again. The second mirror 36 may also include a second surface 128 (e.g., a surface opposite the first surface 126) through which a portion of the beam of light 32 passes to form the beam of therapeutic laser light 40.

The lasing medium 28 may be supported such that the second end 120 of the lasing medium 28 is a fifth distance D5 from the first optically powered surface 122 of the lens 38 (e.g., along a direction parallel to the longitudinal axis 117, such as the longitudinal direction L). According to one embodiment, the fifth distance D5 may be between 115 and 120 mm (e.g., 118.15 mm). The lens 38 may be supported such that the first optically powered surface 122 is a sixth distance D6 from the first surface 126 of the second mirror 36 (e.g., along the direction parallel to the longitudinal axis 117, such as the longitudinal direction L). According to one embodiment, the sixth distance D6 may be between 65 and 70 mm (e.g., 68.76 mm). According to one embodiment, the fifth distance D5 is greater than the sixth distance D6. According to one embodiment, the fifth distance D5 is between 1.5 and 2.0 times greater than the sixth distance D6.

The optical resonator 30 may include a seventh distance D7 measured from the second surface 114 of the first mirror 34 to the first optically powered surface 122 of the lens 38. According to one embodiment the seventh distance D7 may be between 160 and 200 mm (e.g., 180.15 mm). According to one embodiment the seventh distance D7 may be at least 2.5 times greater than the sixth distance D6. The lasing medium 28 may have a length D8 measured from the first end 118 to the second end 120 along the longitudinal axis 117. According to one embodiment the length D8 may be between 60 and 65 mm (e.g., 62.00 mm). According to one embodiment, the fifth distance D5 is between 1.8 and 2.0 times the length D8 (note the drawings, including the distances and radii of curvature, are not drawn to scale).

The optical resonator 30 may include a housing 130 that at least partially encloses an interior cavity 132 within which the first mirror 34, the lasing medium 28, the second mirror 36, the lens 38, or any combination thereof may be at least partially positioned. One or more of the first mirror 34, the second mirror 36, the lens 38, and the lasing medium 28 may be supported within the optical resonator 30 such that one or more of the fifth distance D5, the sixth distance D6, and the seventh distance D7 may be adjusted. According to one embodiment, the first mirror 34, the second mirror 36, the lens 38, and the lasing medium 28 may be supported within the optical resonator 30 such that one or more of the fifth distance D5, the sixth distance D6, and the seventh distance D7 is fixed.

Referring to FIGS. 1, and 12 to 14, the system 10 may include a cooling chamber 140 that regulates the temperature (e.g., removes heat from) the lasing medium 28 during operation of the system 10. The cooling chamber 140 may include an inner wall 142 that forms an inner cavity 144 that receives the lasing medium 28 (e.g., a laser crystal). The inner cavity 144 having a first opening 146 formed in a first terminal end 148 of the cooling chamber 140 and a second opening 150 formed in a second terminal end 152 of the cooling chamber 140.

The cooling chamber 140 may have a length D9 measured from the first terminal end 148 to the second terminal end 152 along a longitudinal axis 154 of the cooling chamber 140 that passes through both the first opening 146 and the second opening 150. As shown, the longitudinal axis 154 may be a central axis of the inner cavity 144. According to one embodiment, the length D9 may be equal to the length D8 of the lasing medium 28.

The cooling chamber 140 may have an outer wall 156 that at least partially encloses the inner wall 142 such that an outer cavity 158 is formed between the inner wall 142 and the outer wall 156. The outer wall 156 may form at least two entry openings 160 that provide passage through the outer wall 156 into the outer cavity 158. The outer wall may also form at least two exit openings 162 that provide passage through the outer wall 156 out of the outer cavity 158.

As shown, each of the at least two entry openings 160 may be positioned closer to the first terminal end 148 than the at least two entry openings 160 are from the second terminal end 152. Also as shown, each of the at least two exit openings 162 may be positioned closer to the second terminal end 152 than the at least two exit openings 162 are from the first terminal end 148. Similarly, each of the at least two entry openings 160 may be closer to the first terminal end 148 than each of the at least two exit openings 162 is from the first terminal end 148.

According to one embodiment, the at least two entry openings 160 may be radially spaced equidistant from one another about the longitudinal axis 154. For example, the cooling chamber 140 may include two entry openings 160 with centers that are spaced 180° from one another about the longitudinal axis 154. As shown, the cooling chamber 140 may include three entry openings 160 with centers that are spaced 120° from one another about the longitudinal axis 154. Similarly, the cooling chamber 140 may include four entry openings 160 with centers that are spaced 90° from one another about the longitudinal axis 154.

According to one embodiment, the at least two exit openings 162 may be radially spaced equidistant from one another about the longitudinal axis 154. For example, the cooling chamber 140 may include two exit openings 162 with centers that are spaced 180° from one another about the longitudinal axis 154. As shown, the cooling chamber 140 may include three exit openings 162 with centers that are spaced 120° from one another about the longitudinal axis 154. Similarly, the cooling chamber 140 may include four exit openings 162 with centers that are spaced 90° from one another about the longitudinal axis 154.

According to one embodiment, there may be an equal number of the at least two entry openings 160 and the at least two exit openings 162 (e.g., two of each, three of each, four of each, etc.). Each of the at least two entry openings 160 may extend through the outer wall 156 along a respective entry opening axis 164, and each of the at least two exit openings 162 may similarly extend through the outer wall 156 along a respective exit opening axis 166. One or more of the respective entry opening axes 164 may be parallel to a respective one of the exit opening axes 166. At least one of the respective entry opening axes 164 may perpendicularly intersect the longitudinal axis 154 of the cooling chamber 140.

In use a coolant (e.g., water) may be pumped through the at least two entry openings 160, into the outer cavity 158 where it absorbs heat from the lasing medium 28 positioned within the inner cavity 144, and exits through the at least two exit openings 162 (as shown by the dashed arrows), thereby removing heat from the cooling chamber 140.

Known cooling chambers that include only one entry opening and only one exit opening (e.g., at the “top” of the cooling chamber) may result in higher temperatures of the lasing medium at the “bottom” of the lasing medium. The cooling chamber 140 with the at least two entry openings 160 and the at least two exit openings 162 may provide improved and/or evenly distributed cooling of the lasing medium 28.

Additionally, as coolant is pumped into the cooling chamber 140 it may create a shockwave or impact force towards/on the lasing medium 28. Such impact force may decrease performance of the system 10. The cooling chamber 140 with the at least two entry openings 160 and the at least two exit openings 162 may reduce the shockwave or impact force imparted upon the lasing medium 28, and the at least two entry openings 160 being evenly spaced radially about the lasing medium 28 may balance any such impact forces imparted upon the lasing medium 28.

According to one embodiment, the outer cavity 158 may be sealed off from a surrounding environment of the cooling chamber 140 except for the at least two entry openings 160 and the at least two exit openings 162. The outer wall 156 may include a radial sidewall 168 that radially surrounds the inner wall 142. The outer wall may further include a first end cap 170 and a second end cap 172 that each include an outer surface that lies in a respective plane that is normal to the longitudinal axis 154 of the cooling chamber 140. The outer wall 156 may be a monolithic, one-piece component, or may include multiple components fastened together.

The cooling chamber 140 (e.g., the inner wall 142, the outer wall 156, or both) may be made of a water resistant material with high thermal conductivity (e.g., bronze).

Referring to FIGS. 1 to 14, a method of operation of the therapeutic laser system 10 may include generating the beam of light 18 from the source laser 16, while the source laser 16 is operating in a first mode, to produce the beam of light 18 with the first beam divergence, focusing the beam of light 18 with the laser focuser 26 and directing the beam of light 18 to the optical resonator 30, impacting the lasing medium 28 with the beam of light 18 to produce the beam of light 32, which has a different wavelength than the beam of light 18, and passing a portion of the beam of light 32 through the second mirror 36 to produce the therapeutic laser light 40.

The method may further include transitioning the source laser 16 to operate in a second mode to produce the beam of light 18 with the second beam divergence, which is different than the first beam divergence, focusing the beam of light 18 with the laser focuser 26 and directing the beam of light 18 to the optical resonator 30, impacting the lasing medium 28 with the beam of light 18 to produce the beam of light 32, which has a different wavelength than the beam of light 18, and passing a portion of the beam of light 32 through the second mirror 36 to produce the therapeutic laser light 40, without moving the source laser 16, any components of the laser focuser 26, and any components of the optical resonator 30 relative to one another. According to one embodiment, the first modes is a continuous wave mode and the second mode is a pulsed mode.

It will be understood by one of skill in the art that the system 10 may include any, up to all, of the source laser 16, the laser focuser 26, the optical resonator 30, and the cooling chamber 140. However, the system 10 does not require each of the source laser 16, the laser focuser 26, the optical resonator 30, and the cooling chamber 140. Any of the source laser 16, the laser focuser 26, the optical resonator 30, and the cooling chamber 140 may be utilized in a laser system other than the laser system 10 as specifically described herein.

The above description of illustrated embodiments, including what is described in the Abstract, is not intended to be exhaustive or to limit the embodiments to the precise forms disclosed. Although specific embodiments of and examples are described herein for illustrative purposes, various equivalent modifications can be made without departing from the spirit and scope of the disclosure, as will be recognized by those skilled in the relevant art.

Many of the methods described herein can be performed with variations. For example, many of the methods may include additional acts, omit some acts, and/or perform acts in a different order than as illustrated or described. The various embodiments described above can be combined to provide further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A laser focuser comprising:
a first lens having a first optical axis, a first optically powered surface, and a second optically powered surface, the first optically powered surface opposite the second optically powered surface along the first optical axis, wherein the first optically powered surface is curved having a first constant radius of curvature, and the second optically powered surface is curved having a second constant radius of curvature;
a second lens having a second optical axis, the second lens positioned with respect to the first lens such that the first optical axis and the second optical axis are collinear, the second lens further having a third optically powered surface and a fourth optically powered surface, the third optically powered surface opposite the fourth optically powered surface along the second optical axis, wherein the third optically powered surface is curved having a third constant radius of curvature, and the fourth optically powered surface is curved having a fourth constant radius of curvature;
a third lens having a third optical axis, the third lens positioned with respect to the first lens and the second lens such that the first optical axis and the third optical axis are collinear, and the second lens is between the first lens and the third lens, the third lens further having a fifth optically powered surface and a sixth optically powered surface, the fifth optically powered surface opposite the sixth optically powered surface along the third optical axis, wherein the fifth optically powered surface is curved having a fifth constant radius of curvature, and the sixth optically powered surface is curved having a sixth constant radius of curvature; and
a fourth lens having a fourth optical axis, the fourth lens positioned with respect to the first lens and the third lens such that the first optical axis and the fourth optical axis are collinear, and the third lens is between the first lens and the fourth lens, the fourth lens further having a seventh optically powered surface and an eighth optically powered surface, the seventh optically powered surface opposite the eighth optically powered surface along the fourth optical axis, wherein the seventh optically powered surface is curved having a seventh constant radius of curvature, and the eighth optically powered surface is curved having an eighth constant radius of curvature,
wherein a first distance measured from the first lens to the second lens along the first optical axis is greater than a second distance measured from the second lens to the fourth lens along the first optical axis.

2. The laser focuser of claim 1 wherein the first distance is at least twice the second distance.

3. The laser focuser of claim 1 wherein the first distance is between 43.29 and 47.27 mm, and the second distance is between 13.57 and 17.54 mm.

4. The laser focuser of claim 1 wherein one or more of the first lens, the second lens, the third lens, and the fourth lens is a spherical lens.

5. The laser focuser of claim 4 wherein the first lens, the second lens, the third lens, and the fourth lens are each spherical lenses.

6. The laser focuser of claim 1 wherein the first optically powered surface is convex and the second optically powered surface is convex.

7. The laser focuser of claim 6 wherein the first optically powered surface is cylindrical having an infinite radius of curvature measured within a plane perpendicular to the first constant radius of curvature.

8. The laser focuser of claim 6 wherein the second optically powered surface is cylindrical having an infinite radius of curvature measured within a plane perpendicular to the second constant radius of curvature.

9. The laser focuser of claim 1 wherein the second lens is oriented such that the third optically powered surface faces towards the first lens and the fourth optically powered surface faces towards the third lens, the third optically powered surface is convex, and the fourth optically powered surface is concave.

10. The laser focuser of claim 9 wherein the third optically powered surface is cylindrical having an infinite radius of curvature measured within a plane perpendicular to the third constant radius of curvature.

11. The laser focuser of claim 9 wherein the fourth optically powered surface is cylindrical having an infinite radius of curvature measured within a plane perpendicular to the fourth constant radius of curvature.

12. The laser focuser of claim 1 wherein the third lens is oriented such that the fifth optically powered surface faces towards the second lens and the sixth optically powered surface faces towards the fourth lens, the fifth optically powered surface is convex, and the sixth optically powered surface is convex.

13. The laser focuser of claim 12 wherein the fifth optically powered surface is cylindrical having an infinite radius of curvature measured within a plane perpendicular to the fifth constant radius of curvature.

14. The laser focuser of claim 12 wherein the sixth optically powered surface is cylindrical having an infinite radius of curvature measured within a plane perpendicular to the sixth constant radius of curvature.

15. The laser focuser of claim 1 wherein the fourth lens is oriented such that the seventh optically powered surface faces towards the third lens, the seventh optically powered surface is convex, and the eighth optically powered surface is concave.

16. The laser focuser of claim 15 wherein the seventh optically powered surface is cylindrical having an infinite radius of curvature measured within a plane perpendicular to the seventh constant radius of curvature.

17. The laser focuser of claim 15 wherein the eighth optically powered surface is cylindrical having an infinite radius of curvature measured within a plane perpendicular to the eighth constant radius of curvature.

18. The laser focuser of claim 1 wherein the first lens, the second lens, the third lens, and the fourth lens are each toroidal lenses.

19. The laser focuser of claim 1, further comprising a housing at least partially enclosing each of the first lens, the second lens, the third lens, and the fourth lens.

20. An optical resonator comprising:
a first mirror having a first surface that is transmissive of light within a first range of wavelengths, the first mirror having a second surface, opposite the first surface, that is reflective of light within a second range wavelengths;
a laser crystal positioned with respect to the first mirror such that a longitudinal axis of the laser crystal intersects the first mirror;
a lens positioned with respect to the first mirror and the laser crystal such that the longitudinal axis of the laser crystal intersects the lens and the laser crystal is between the first mirror and the lens; and
a second mirror having a third surface that reflects a majority of light with the second wavelength that contacts the first surface and transmits a portion of the light with the second wavelength that contacts the first surface through the third surface,
wherein a first distance measured from the laser crystal to the lens along the longitudinal axis is greater than a second distance measured from the lens to the second mirror along the longitudinal axis.

21. The optical resonator of claim 20 wherein the first distance is between 1.5 and 2.0 times greater than the second distance.

22. The optical resonator of claim 20 wherein a third distance measured from the first mirror to the lens is at least 2.5 times greater than the second distance.

23. The optical resonator of claim 20 wherein the laser crystal has a length measured along the longitudinal axis, and the first distance is between 1.8 and 2.0 times the length.

24. The optical resonator of claim 20 wherein the laser crystal is a laser crystal doped with holmium, thulium, or both holmium and thulium.

25. The optical resonator of claim 24 wherein the laser crystal is a yttrium-aluminum-garnet laser crystal doped with holmium, thulium, or both holmium and thulium.

26. The optical resonator of claim 20 wherein the longitudinal axis is a central axis of the laser crystal along which the laser crystal is elongated.

27. The optical resonator of claim 20 wherein the first mirror includes a mirror optical axis, and the first mirror is positioned with respect to the laser crystal such that the mirror optical axis is collinear with the longitudinal axis.

28. The optical resonator of claim 27 wherein the lens includes a lens optical axis, and the lens is positioned with respect to the laser crystal such that the lens optical axis is collinear with the longitudinal axis.

29. The optical resonator of claim 20 wherein the lens includes a lens optical axis, and the lens is positioned with respect to the laser crystal such that the lens optical axis is collinear with the longitudinal axis.

30. The optical resonator of claim 20 wherein the first range of wavelengths and the second range of wavelengths are mutually exclusive.

31. The optical resonator of claim 30 wherein the first range of wavelengths is between 700 and 900 nm, and the second range of wavelengths is between 1800 and 2200 nm.

32. The optical resonator of claim 20, further comprising:
a cooling chamber including:
an inner wall that forms an inner cavity that receives the laser crystal; and
an outer wall that forms an outer cavity between the inner wall and the outer wall, wherein the outer wall forms at least two entry openings that provide passage into the outer cavity and at least two exit openings that provide passage out of the outer cavity.

33. The optical resonator of claim 32 wherein the cooling chamber has a cooling chamber length that is measured from a first terminal end of the cooling chamber to a second terminal end of the cooling chamber along a direction parallel to a longitudinal axis of the cooling chamber.

34. The optical resonator of claim 33 wherein the longitudinal axis of the cooling chamber is a central axis of the inner cavity, and when the laser crystal is received within the inner cavity the longitudinal axis of the cooling chamber is collinear with the longitudinal axis of the laser crystal.

35. The optical resonator of claim 33 wherein:
each of the at least two entry openings are positioned closer to the first terminal end than the at least two entry openings are from the second terminal end; and
each of the at least two exit openings are positioned closer to the second terminal end than the at least two exit openings are from the first terminal end.

36. The optical resonator of claim 35 wherein the at least two entry openings are radially spaced equidistant from one another about the longitudinal axis of the cooling chamber.

37. The optical resonator of claim 35 wherein the at least two exit openings are radially spaced equidistant from one another about the longitudinal axis of the cooling chamber.

38. The optical resonator of claim 32 wherein there is an equal number of the at least two entry openings and the at least two exit openings.

39. The optical resonator of claim 38 wherein each of the at least two entry openings extends through the outer wall along a respective entry opening axis, each of the at least two exit openings extends through the outer wall along a respective exit opening axis, and each of the respective entry opening axes is parallel to at least one of the respective exit opening axes.

40. The optical resonator of claim 32 wherein the outer cavity is only accessible through the at least two entry openings and the at least two exit openings.

41. The optical resonator of claim 20, further comprising a housing at least partially enclosing each of the first mirror, the laser crystal, the lens, and the second mirror.

42. A laser crystal cooling chamber comprising:
an inner wall that forms an inner cavity that receives a laser crystal, the inner cavity having a first opening formed in a first terminal end of the cooling chamber and a second opening formed in a second terminal end of the cooling chamber, wherein the cooling chamber has a cooling chamber length that is measured from the first terminal end to the second terminal end along a longitudinal axis of the cooling chamber that passes through both the first opening and the second opening; and
an outer wall that at least partially encloses the inner wall such that an outer cavity is formed between the inner wall and the outer wall, the outer wall forms at least two entry openings that provide passage through the outer wall into the outer cavity, and the outer wall forms at least two exit openings that provide passage through the outer wall out of the outer cavity,
wherein each of the at least two entry openings are positioned closer to the first terminal end than the at least two entry openings are from the second terminal end, and each of the at least two exit openings are positioned closer to the second terminal end than the at least two exit openings are from the first terminal end.

43. The laser crystal cooling chamber of claim 42 wherein the longitudinal axis of the cooling chamber is a central axis of the inner cavity.

44. The laser crystal cooling chamber of claim 42 wherein the at least two entry openings are radially spaced equidistant from one another about the longitudinal axis of the cooling chamber.

45. The laser crystal cooling chamber of claim 42 wherein the at least two exit openings are radially spaced equidistant from one another about the longitudinal axis of the cooling chamber.

46. The laser crystal cooling chamber of claim 42 wherein there is an equal number of the at least two entry openings and the at least two exit openings.

47. The laser crystal cooling chamber of claim 46 wherein each of the at least two entry openings extends through the outer wall along a respective entry opening axis, each of the at least two exit openings extends through the outer wall along a respective exit opening axis, and each of the respective entry opening axes is parallel to at least one of the respective exit opening axes.

48. The laser crystal cooling chamber of claim 47 wherein at least one of the respective entry opening axes perpendicularly intersects the longitudinal axis of the cooling chamber.

49. The laser crystal cooling chamber of claim 42 wherein the outer cavity is only accessible through the at least two entry openings and the at least two exit openings.

50. The laser crystal cooling chamber of claim 42 wherein the outer wall includes a radial sidewall that radially surrounds the inner wall, and the outer wall include a first end cap and a second end cap, the first end cap and the second end cap each including an outer surface that lies in a respective plane that is normal to the longitudinal axis of the cooling chamber.

51. The laser crystal cooling chamber of claim 42 wherein at least one of the inner wall, the outer wall, or both the inner wall and the outer wall is made of bronze.

52. A laser system comprising:
a laser diode;
a laser focuser of having:
a first lens having a first optical axis, a first optically powered surface, and a second optically powered surface, the first optically powered surface opposite the second optically powered surface along the first optical axis, wherein the first optically powered surface is curved having a first constant radius of curvature, and the second optically powered surface is curved having a second constant radius of curvature;
a second lens having a second optical axis, the second lens positioned with respect to the first lens such that the first optical axis and the second optical axis are collinear, the second lens further having a third optically powered surface and a fourth optically powered surface, the third optically powered surface opposite the fourth optically powered surface along the second optical axis, wherein the third optically powered surface is curved having a third constant radius of curvature, and the fourth optically powered surface is curved having a fourth constant radius of curvature;
a third lens having a third optical axis, the third lens positioned with respect to the first lens and the second lens such that the first optical axis and the third optical axis are collinear, and the second lens is between the first lens and the third lens, the third lens further having a fifth optically powered surface and a sixth optically powered surface, the fifth optically powered surface opposite the sixth optically powered surface along the third optical axis, wherein the fifth optically powered surface is curved having a fifth constant radius of curvature, and the sixth optically powered surface is curved having a sixth constant radius of curvature; and
a fourth lens having a fourth optical axis, the fourth lens positioned with respect to the first lens and the third lens such that the first optical axis and the fourth optical axis are collinear, and the third lens is between the first lens and the fourth lens, the fourth lens further having a seventh optically powered surface and an eighth optically powered surface, the seventh optically powered surface opposite the eighth optically powered surface along the fourth optical axis, wherein the seventh optically powered surface is curved having a seventh constant radius of curvature, and the eighth optically powered surface is curved having an eighth constant radius of curvature,
wherein a first distance measured from the first lens to the second lens along the first optical axis is greater than a second distance measured from the second lens to the fourth lens along the first optical axis;
an optical resonator having:
a first mirror having a first surface that is transmissive of light within a first range of wavelengths, the first mirror having a second surface, opposite the first surface, that is reflective of light within a second range wavelengths;
a laser crystal positioned with respect to the first mirror such that a longitudinal axis of the laser crystal intersects the first mirror;
a lens positioned with respect to the first mirror and the laser crystal such that the longitudinal axis of the laser crystal intersects the lens and the laser crystal is between the first mirror and the lens; and
a second mirror having a third surface that reflects a majority of light with the second wavelength that contacts the first surface and transmits a portion of the light with the second wavelength that contacts the first surface through the third surface,
wherein a first distance measured from the laser crystal to the lens along the longitudinal axis is greater than a second distance measured from the lens to the second mirror along the longitudinal axis; and
a laser crystal cooling chamber having:
an inner wall that forms an inner cavity that receives a laser crystal, the inner cavity having a first opening formed in a first terminal end of the cooling chamber and a second opening formed in a second terminal end of the cooling chamber, wherein the cooling chamber has a cooling chamber length that is measured from the first terminal end to the second terminal end along a longitudinal axis of the cooling chamber that passes through both the first opening and the second opening; and
an outer wall that at least partially encloses the inner wall such that an outer cavity is formed between the inner wall and the outer wall, the outer wall forms at least two entry openings that provide passage through the outer wall into the outer cavity, and the outer wall forms at least two exit openings that provide passage through the outer wall out of the outer cavity,
wherein each of the at least two entry openings are positioned closer to the first terminal end than the at least two entry openings are from the second terminal end, and each of the at least two exit openings are positioned closer to the second terminal end than the at least two exit openings are from the first terminal end,
wherein the laser focuser is positioned between the laser diode and the optical resonator, and the laser crystal is positioned within the inner cavity.

53. The laser system of claim 52, further comprising:
an enclosure that encloses each of the laser diode, the laser focuser, the optical resonator, and the laser crystal cooling chamber.
